(19) United States
(12) Patent Application Publication
(10) Pub. No.: US 2019/0374351 A1
(43) Pub. Date: Dec. 12, 2019

(54) Transplantation Methods and Devices for Bone Surface Contouring

(71) Applicant: Joint Restoration Foundation, Inc, d/b/a JRF Ortho, Centennial, CO (US)

(72) Inventors: Timothy Mologne, Appleton, WI (US); Daniel Cerundolo, Weymouth, MA (US); David Wilke, Canton, GA (US)

(21) Appl. No.: 16/002,662

(22) Filed: Jun. 7, 2018

(51) Int. Cl. A61F 2/46; A61F 2/28; A61B 17/16

(52) U.S. Cl. CPC A61F 2/4644; A61F 2/28; A61F 2/4657; A61B 17/1635; A61F 2002/4645; A61F 2310/00359; A61F 2002/4687; A61F 2002/4663; A61F 2002/2839

(57) ABSTRACT

A surface contour gauge is provided for use in surgical transplantation procedures. Surface contour gauges provide quick and efficient surface contour matches between a patient's bone defect and a prepared allograft plug.

### TECHNICAL FIELD

The described embodiments relate generally to surgical transplant procedures. More particularly, the present embodiments relate to methods and instruments for matching the three dimensional (3D) surface contour of a patient's excised osteochondral tissue to the 3D surface of its replacement allograft plug.

### BACKGROUND OF THE INVENTION

Allograft bone/cartilage transplantation is a desired surgical procedure for a number of orthopedic procedures. Allograft-based transplantations are typically used to fill larger bone defects caused by, for example, traumatic injury, cancer, or bone infection. In such procedures, an allograft is harvested from a donor source, typically a solid cadaver bone, and inserted in the bone defect. The transplanted allograft fills the defect and acts as a scaffold for bone growth and facilitated healing.

Allograft bone/cartilage, or bone from a cadaver, has become more accessible for use in bone transplantation due to improved procedures for obtaining, screening, disinfecting and storing cadaver bone. In this light, the use of fresh cadaver bone as an osteoarticular allograft source is showing more promise than the use of frozen bone, and surgical procedures using this type of fresh source material are becoming the normative. At present, storage of fresh cadaver bone for use in surgical procedures is recommended to be around 24 to 28 days, meaning that the use and shelf life of cadaver bones is limited, and presents a hurdle to efficient use of the material.

Use of an osteochondral allograft for filling a target bone defect is typically tied to matching the size and contour surfaces of the two bones. In this regard, matching can be accomplished by a surgeon who scans the allograft for a reasonable surface match with the recipient bone. Certain sizing instruments can be used to roughly match the allograft plug to the defect. Further, by using experience and similar allograft sources as the target recipient bone, a surgeon can facilitate matching an allograft 3D surface to the surrounding bone at the defect site. In such cases, the surgeon uses his or her experience to find a site on the cadaver bone, and size the identified allograft to the excised defect site to facilitate healing.

Proper contour matching of the surface of an osteoarticular allograft to the surface of a target bone defect is extremely important for proper healing and transplantation success. As allograft bone transplantation procedures have developed, it has been recognized that mismatched articular surfaces (allograft to recipient) often result in significantly higher risk for recipient bone damage and transplant failure. In addition, due to the limited amount of allograft source material, efficient use of these fresh osteoarticular sources is of growing importance. Expanded use of allograft sources for target defects and more precise surface contour matching is required in the art.

The present invention is directed toward overcoming one or more of the problems discussed above.

### BRIEF DESCRIPTION OF THE DRAWINGS

FIG. 1 is a schematic view of a surface contour gauge engaged to a bone defect in accordance with embodiments herein.

FIG. 2 is an illustrative schematic view of the central hub and positioning pins in accordance with embodiments herein.

FIG. 3 is a schematic view of a surface contour gauge in accordance with embodiments herein.

FIG. 4 is a schematic view of the cartilage being scored with a cutter after the bone defect surface was identified using the surface contour gauge of the present invention.

FIG. 5 is a schematic view of a surface contour gauge and corresponding ring guide for allograft plug preparation in accordance with embodiments herein

FIG. 6 is a schematic view of surface matching using the surface contour gauge and ring guide in accordance with embodiments herein.

FIG. 7 is a schematic view of positioning of a coring reamer in relation to a ring guide in accordance with embodiments herein.

FIG. 8 is a flow chart in accordance with embodiments herein.

FIG. 9 is an alternative flow chart in accordance with embodiments herein.

### SUMMARY OF THE INVENTION

Embodiments in accordance with the disclosure include methods and devices for matching the 3D surface contour of excised osteochondral tissue to the 3D surface contour of an allograft plug. In general, the ability to replace an excised bone defect with an allograft plug, where the allograft plug has a similar, or the same, surface contour provides improved outcomes for the transplantation. Bone surface contour matching is accomplished through the use of a surface contour gauge (or gauge herein), a device that allows a surgeon to make a simple negative contour profile of a bone defect and replicate that contour by screening allograft sources for a match.

As such, in one embodiment, a gauge for accomplishing a bone surface match is provided. The gauge includes a central hub having a top surface and an opposing bottom surface, with a uniform amount of material therebetween. An alignment tube is operatively integrated into the top surface of the central hub and forms an opening through the central hub (the alignment tube for receiving an attachment pin that attaches the central hub to an underlying bone surface, and thereby providing orientation). A plurality of positioning pins extend through a matching number of openings in the central hub, such that the positioning pins can be traversed within the openings. Positioning pins are traversed through the central hub, which acts as a platform for the pins, until each touches the underlying bone surface. Positioning pins can be advanced and retracted as needed by a user of the device. The distances that each positioning pin extends below the central hub provides a contour of the underlying bone surface.

In some aspects the central hub does not include an alignment tube, and only has an opening defined for receiving an attachment pin. In some aspects, the gauge includes at least 4 positioning pins to provide a bone surface contour, and most typically, at least 6 positioning pins or at least 8 positioning pins. Positioning pins can be arrayed and received around the perimeter of the central hub, and are typically in a pattern that covers at least the 3, 6, 9 and 12 o'clock positions of a clock face.

Embodiments herein also include methods for using the gauge to match two bone surfaces. Initially, a first bone surface contour is identified using the gauge. The gauge, having the first bone surface contour set, is then used to screen a second bone surface. The gauge can be walked over the second bone surface until the positioning pins substantially match the profile of the first bone surface. Not all positioning pins must exactly match the surface of the second bone surface, and in some cases, 3 out of 4 may be sufficient, or 4 out of 6, for example, depending on the need and availability of source material.

In aspects of the method, a first bone surface is a bone surface having a bone defect in a patient. The surgeon exposes the bone defect and determines the surface contour using the contour gauge described herein. Typically, the gauge is attached to the underlying bone at the defect by driving a pin into and through the central tube. The gauge is set for identifying a bone surface match that will sit in the newly formed cavity. An allograft source is obtained and screened by the surgeon (or other health care worker) using the set gauge. Once a match is obtained, an allograft plug having the same size and relative bone surface is cut out and transplanted into the cavity formed at the patient's defect site. A bone cavity is then formed in the patient's condyle. As is described in greater detail below, the allograft plug has the same relative surface profile as the excised bone defect, allowing for better use of the allograft source material, and providing the patient with a greater opportunity to properly heal.

Finally, the disclosure herein provides kits for transplanting a contour matched allograft plug to a condylar defect. Kits can include a contour gauge capable of matching the condylar defect surface to a donor bone surface; a ring guide for marking the site for allograft plug removal and for receiving a coring reamer for fine tuning the size of the allograft plug. Kits may also include a trimming device for fine tuning the size of the allograft plug. In some aspects, the kit can include steinmann pins, instructions and other useful materials for a bone transplantation surgery.

Other embodiments in accordance with the disclosure herein will become apparent from a review of the ensuing detailed description.

### DETAILED DESCRIPTION OF THE INVENTION

Reference will now be made in detail to representative embodiments illustrated in the accompanying drawings. It should be understood that the following descriptions are not intended to limit the embodiments to one preferred embodiment. To the contrary, they are intended to cover alternatives, modifications, and equivalents as can be included within the spirit and scope of the described embodiments as defined in the appended claims.

The following disclosure relates to methods and devices useful in the surgical transplantation of osteoarticular allografts to targeted bone defects, and in particular, to matching the size and 3D surface contour of an osteoarticular allograft to the size and 3D surface of an excised bone defect. A size and surface matched allograft, where the edges of the allograft substantially align with the edges of the prepared cavity in the target bone (patient's bone) has a significantly improved capacity to integrate with the recipient bone, and facilitate defect healing. Where the size or surface contour of the allograft and targeted bone defect do not match, or only partially match, integration and healing of the allograft is diminished, and the likelihood of re-injury significantly increased.

Embodiments in accordance with the present invention relate to a surface contour gauge (referred to herein as a “contour gauge” or “gauge”) for matching a 3D surface contour of an excised lesion to a 3D surface contour of a osteoarticular allograft. The gauge also allows for accurate size matching of the excised lesion and allograft at the same time.

The gauge expands the utility of osteoarticular allograft source material as well, such that transplantation is not limited by Left/Right orientation of the cadaver bone, cadaver bone type, or the recipient defect location in relation to the cadaver bone. Rather, embodiments of the present disclosure allow for allograft sources to come from any solid bone source where the size and surface contours can be matched, and thus allows for a significant expansion of fresh cadaver bone utility. Further, embodiments herein allow for a more consistent and efficient use of allograft source material, as compared to a surgeon's best guess, where gauge assisted allograft plug formation is prepared with substantially matching size and 3D surface contour. In this manner, an allograft as identified using the embodiments herein, can be harvested from available fresh cadaver bone.

Aspects of the disclosure include a device for accurately matching a first bone surface to a second bone surface, such that the first and second bone surfaces have relatively similar 3D surface contours. In some aspects, the first bone surface is a surface of a bone lesion in need of resurfacing, and the second bone surface is a surface of an osteoarticular allograft for replacement of the first bone surface. As an aspect of the surface contour match, a measured size of the allograft is also obtained during the transplantation procedure.

As described herein, matching of 3D surface contours refers to the general overall form of two bone surfaces, but is particularly directed toward the 3D contour surface along the edge where the allograft will be contiguous with the recipient bone. For example, a 3D surface contour match is one in which the edges of the two bone surfaces (allograft and excised defect), when substituted for each other, abut surrounding bone and show minimal displacement, and in particular, vertical displacement, with existing non-defect bone.

As such, aspects include methods for measuring the size and 3D surface contour of a bone defect or lesion, and replacing that bone defect with an osteoarticular allograft having a correct size and matched bone surface. Aspects herein can also include kits that include the components necessary to identify and prepare an osteoarticular allograft for use in transplantation to a target bone defect, including the gauge (with ring guide), a cartilage guide, a flat blade for defect removal, steinmann pins, and a coring reamer. In some aspects kits may also include trimming devices and instructions on how to prepare a 3D matching allograft plug for a bone defect.

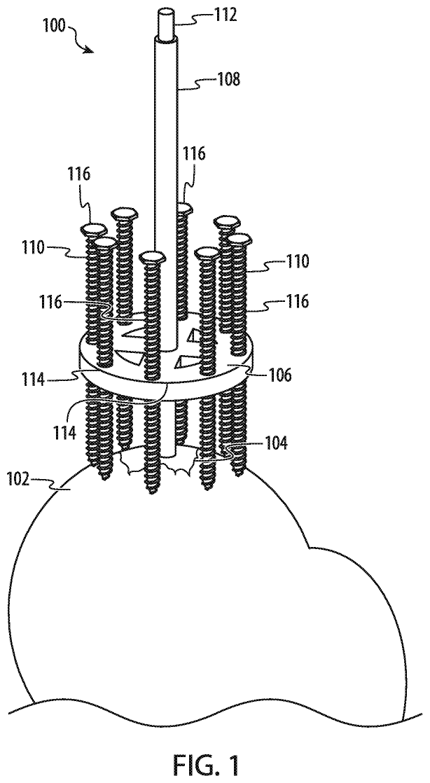

These and other embodiments are discussed below with reference to FIGS. 1-9. However, those skilled in the art will readily appreciate that the detailed description given herein with respect to these Figures is for explanatory purposes only and should not be construed as limiting.

FIG. 1 schematically illustrates a gauge 100 in accordance with embodiments herein attached to a bone surface 102 having a defect 104 in need of repair. The gauge 100 includes a central hub 106 with integrated alignment tube 108, and a plurality of contour positioning pins 110. As is described in greater detail below, the central hub 106 acts as a platform for orienting and setting a contour profile for the underlying bone surface 102. An attachment pin 112 is positioned within the alignment tube 108 to attach and orient the central hub 106 above a bone surface 102. In some embodiments, the alignment pin is attached to the defect such that the central hub is at a right angle to the attachment point. In typical embodiments, the attachment pin contacts the bone within the defect site 104. Once the central hub 106 is positioned, the plurality of contour positioning pins 110 are extended through the central hub 106 to the bone surface 102 to provide a negative profile of the underlying bone surface. A negative profile herein can be set by as few as four points, and more typically by six, eight, or twelve points. Each point is established as a distance between the central hub and the bone surface as measured by each positioning pin. In addition, central hubs may include an indentation or mark to identify an orientation position, for example, a position that would be aligned with 12 o'clock of a clock face. The central hub 106 may have more that one “orbits” of holes to accept the positioning pins 110. The diameters of these orbits would match the diameters of the defects (not shown). However, aspects herein can include a central hub that defines one or more orbits, two or more orbits, or three or more orbits, such that one central hub can be used to profile a number of different sized defects.

As such, a negative bone surface profile herein requires at least four or more positioning pins 110 spaced around the outer edge 114 of the central hub 106, so for example, at least one positioning pin 110 at the numbers 3, 6, 9 and 12 o'clock of a clock face 116 (forming an orbit of holes). Other embodiments, include five or more, six or more, seven or more, eight or more, nine or more, ten or more, eleven or more, twelve or more, thirteen or more, fourteen or more, fifteen or more, sixteen or more, seventeen or more, eighteen or more, and the like. Positioning pins can be equally spaced from each other or positioned anywhere through the central hub, as long as a negative surface profile can be generated to establish correct surface positioning of the allograft. Having at least four points of contour around a defect site allows for a substantial match to an allograft source and ensures that the defect cavity and allograft plug will abut at least at those points. Additional positioning pin points of contour allow for closer and closer matches, although the inventors herein have found significant utility where at least four points are used. In addition, an appropriate 3D contour is one where the prepared allograft plug has edges that abut the edges of the recipient bone. Alternations of contour beyond the edges are helpful, but of lesser concern.

Once the gauge 100 has an established negative bone surface profile, it is removed from the bone surface having the defect, and used to screen and identify corresponding or acceptable bone surfaces on allograft source material. The allograft source can be any acceptable solid bone from a cadaver that potentially has matches for the negative bone surface profile, and that has an acceptable bone density for the defect site.

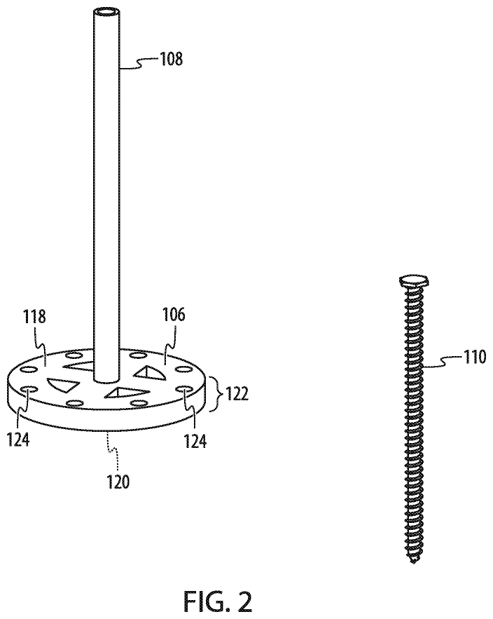

Referring to FIG. 2, the central hub 106 has a top surface 118 and an opposing bottom surface 120. The top surface and bottom surface are typically flat and separated by an uniform thickness of material 122. Typical central hub thicknesses are from about 2 mm to about 20 mm, and more typically from about 10 mm to 15 mm.

As shown in FIG. 2, the central hub 106 can be circular, but could also be oval, or other useful shape, e.g., square, triangular, rectangular, octagonal, etc. The alignment tube 108 generally extends off of the top surface 118 at a right angle, and can be centered in the central hub. An alignment tube 108 can be from about 2.0 to 3.0 inches in length, and more typically from about 2.25 to about 2.75 inches in length. The central hub, including the alignment tube, can be disposable or non-disposable, prepared from, for example, steel, steel alloys, polymers, and the like. Central hub diameter size can be varied and based on the size of defect to be filed, i.e., the size of surface contour that must be matched. In alternative embodiments, the central hub does not require an alignment tube, but rather has a centrally located opening to directly receive an attachment pin. In both aspects, the central hub can be slid onto and off of an attachment pin, for ease of use.

Central hub embodiments can also be of a solid and uniform thickness of material and define multiple orbits of holes, or the central hub can be of a “wagon-wheel” design with only minimal orbits of holes drilled in the outer periphery of the wheel. This “wagon-wheel” embodiment is shown in FIG. 2.

A series of peripherally located openings or holes 124 are situated through the central hub for receiving positioning pins 110. Openings in the central hub 106 correspond to the number of positioning pins required to map or profile the underlying bone surface. In some aspects, openings 124 are uniformly located along the peripheral edge of the central hub. For example, an opening for receiving a contour positioning pin, at 12, 3, 6 and 9 o'clock of a clock face. In other aspects, two sets of openings, can be positioned in the central hub, a first set along the peripheral edge, and a second set at a predetermined radius from the alignment tube (not shown). Depending on the size of the central hub, additional openings can be positioned. Openings can be threaded, where the positioning pins are threaded pins or screws. As noted above, various sets of openings can be included on a single central hub to allow for uses on any number of sized defects, or can be used to allow for additional profile data points to further allow for precise 3D surface contouring. In addition, positioning pins can be advanced or retracted within the central hub, so that identifying underlying surface contours is quickly mastered.

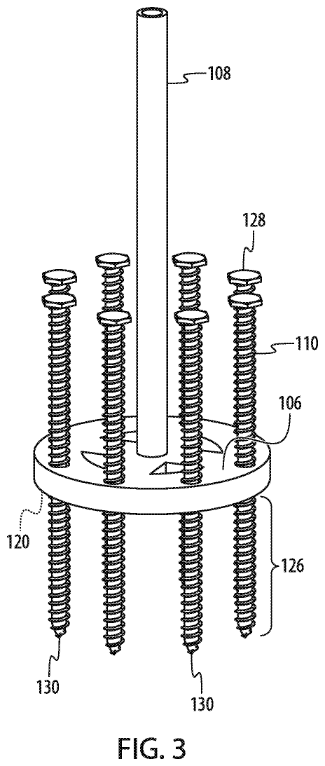

As shown in FIG. 3, positioning pins 110 can be threaded or smooth (not shown), depending on the type of opening in the central hub 106. Positioning pins are typically of a length 126 that they are received in the openings and can be extended to at least 10 mm beyond the central hub bottom surface 120. In some embodiments, the positioning pins 110 are at least 12 mm in length, at least 13 mm in length, at least 14 mm in length, at least 15 mm in length, at least 16 mm in length, at least 17 mm in length, at least 18 mm in length, at least 19 mm in length, at least 20 mm in length, and the like. As discussed in greater detail below, a positioning ring can be layered below a central hub, such that the positioning pins are extended to a length beyond the width of the positioning ring, i.e., the positioning pins extend below the bottom surface of the positioning ring. Positioning pin heads 128 can include indents for actuating the pins relative to the central hub or can include a surface for the user to press against to slide the pin relative to the central hub. The distal end 130 of each positioning pin can be pointed for precise contact with the underlying bone surface.

The alignment tube 108 is hollow and designed to receive an attachment pin, for example, a steinmann pin, to orient and attach the central hub to an underlying bone surface of interest.

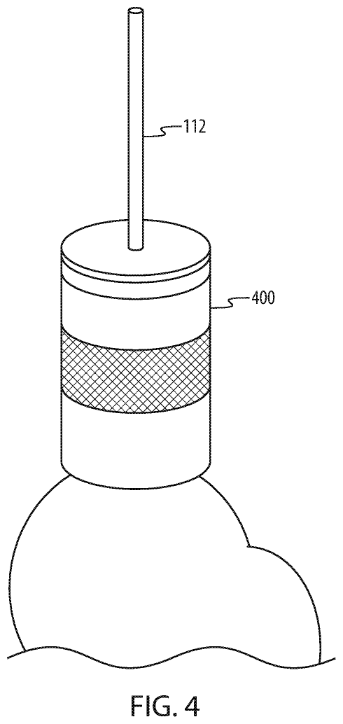

Once a negative contour profile has been set by the gauge 100, the defect 104 in need of repair is scored for defect removal. As shown in FIG. 4, a cutter/guide 400 is oriented around the attachment pin 112 (note that the gauge has been pulled off of the defect site, leaving only the attachment pin in the defect).

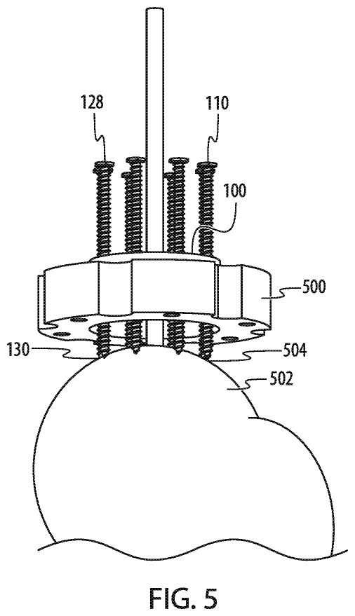

Defect removal can be performed by a flat blade, or other like device, for forming an appropriate cavity at the scored site. Depending on the depth of the defect, the flat blade can be advanced to form an appropriate cavity. In typical aspects, the cavity has a depth of from about 3 mm to about 12 mm, and more typically, from about 3 mm to about 6 mm. The bone surface having the defect has been measured for contour and a sized cavity prepared. Preparation of a matching sized surface contour allograft plug is now shown in FIGS. 5-7. Note also that the depth of the cavity can also be identified at a number of points along the edge, for example, where the cavity has a non-uniform depth, identify a depth at a 12, 9, 6 and 3 o'clock positions of a clock face.

FIG. 5 shows a gauge 100 operatively attached to a ring guide 500, such that the ring guide 500 is used to form an allograft plug (not shown) once a size and surface match on the allograft source 502 is identified. The gauge 100 having the set positioning pins 110 for the defect site, is encompassed by the ring guide 500 and “walked” over an allograft source surface 504 to find the most appropriate mating contour. In an alternative aspect, the gauge can be walked over the allograft surface to identify the proper surface match, prior to the ring guide being combined or assembled with gauge.

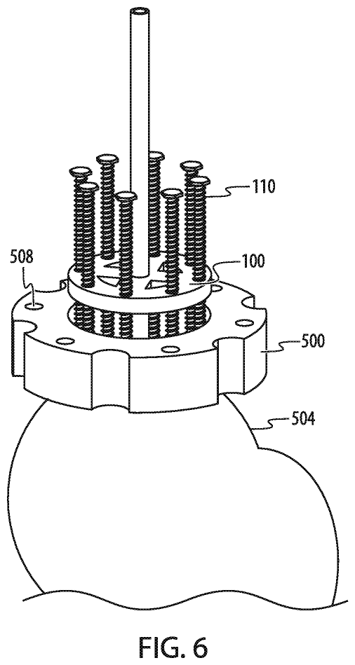

Mating contours or matching contours show substantial matching when the positioning pins touch the allograft surface. However, not all positioning pins may exactly match or touch an allograft surface, so some discrepancy is tolerated. As discussed above, a minimum of 4 points or positioning pins are used to identify the matching surface. The use of 4 positioning pins sets a negative profile on the gauge. Once the gauge is oriented over a matching allograft surface 504, as shown in FIG. 6, one or more steinmann pins 506, or other like attachment devices, can be drilled through openings 508 in the ring guide 500 and into the underlying condyle. Also note that the gauge does not have to be attached to the underlying allograft, where the surgeon does not feel it necessary to have that type of stability.

Alternatively, the ring guide may also be secured in place by an articulating arm with a locking mechanism (not shown). The articulating arm can be mounted to a platform or other useable workstation, as deemed appropriate by the surgeon.

Note that ring guide 500 is positioned around the periphery of the matched allograft plug, so that the ring guide attachment does not damage the allograft surface. The ring guide and gauge are typically sized to match a coring reamer, such that the coring reamer will sit inside and be oriented by the ring guide (once the gauge has been removed).

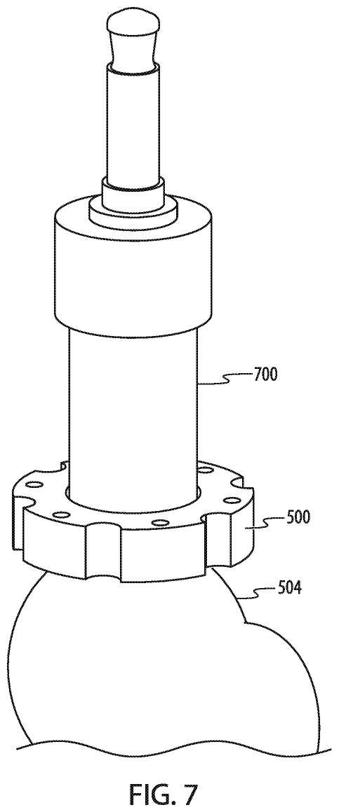

As shown in FIG. 7, the gauge is removed from the allograft attached ring guide 500 and a coring reamer 700, or other like device, is positioned. The coring reamer is advanced to procure the allograft plug for the transplant. The ring guide can now be removed, and an allograft plug released from the allograft source. The allograft plug has a matched surface contour to the cavity formed at the bone defect site. Typical plugs are cut to a depth of about 6-12 mm, although other depths are contemplated. In some cases, depth measurements can be made from the defect cavity to further aid in the removal of tissue from the plug. In some aspects, trimming tools can be used to remove excess material where a depth of the allograft plug does not meet the depth of the defect cavity.

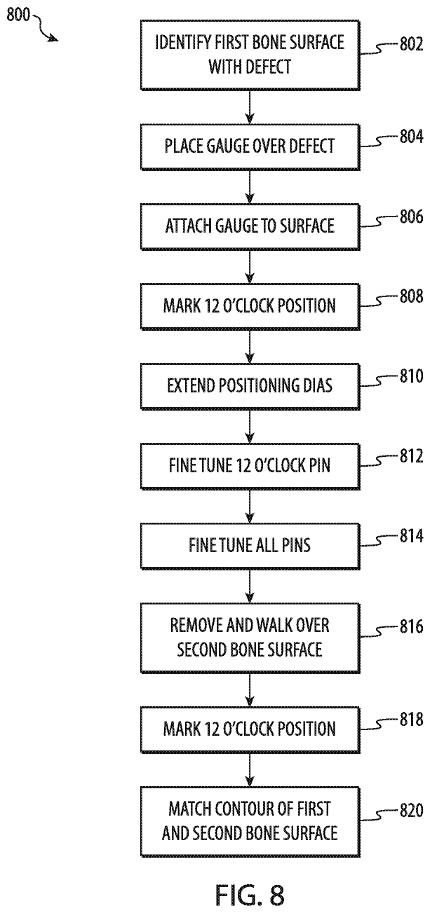

FIG. 8 shows a flow chart of a method for matching 3D bone surfaces of a first bone surface to a second bone surface 800. In a first operation 802, a first bone surface in need of a surface profile is identified in a patient. Typical bone surfaces have a condyle defect. In operation 804, a contour gauge in accordance with embodiments herein is placed directly over the condyle defect. Since defect sizes and bone surface geometries differ, different sized contour gauges can be used. An appropriately sized contour gauge is positioned over the condyle defect. In operation 806, an attachment pin, received in the alignment tube, is advanced into the bone surface at the defect to attach the contour gauge to the bone surface. Typical attachment pins are steinmann pins. In operation 808, the 12 o'clock position is marked on the condyle, relative to the contour gauge.

In operation 810, a series of four or more positioning pins are extended from the central hub of the gauge to the underlying bone surface to form a bone surface contour profile. Note that 5, 6, 7, 8 or more positioning pins may be used to obtain the bone surface contour profile. The positioning pins must extend below the central hub to a distance of at least as thick as the ring guide prior to an initial setting. In operation 812, the positioning pins are further fine tuned into position to provide a final surface profile, or a negative profile of the underlying surface. In this light, one of the positioning pins is aligned with the 12 o'clock position on the condyle to provide an orientation for the contour gauge. In operation 814, the positioning pins having been fine-tuned are examined to identify surface contact and ensure that all pins are in a final position. This is particularly important, touching the condyle surface, for the positioning pin at the 12 o'clock location. In some instances the pins may be advanced and retracted to ensure that all the pins are making contact with the condyle surface.

In operation 816, the contour gauge is removed from the first bone surface, and walked over various aspects of a second bone surface. The negative bone surface alignment of extended pins are slid or moved until a match is identified. Once a position is selected, the 12 o'clock and 6 o'clock pins are marked on the condyle. Note that other pin positions can be used, as long as the markings are consistent with identifying the bone surface contour of the patient. In operation 818, the fist bone surface and second bone surface are identified as having substantially matching surfaces.

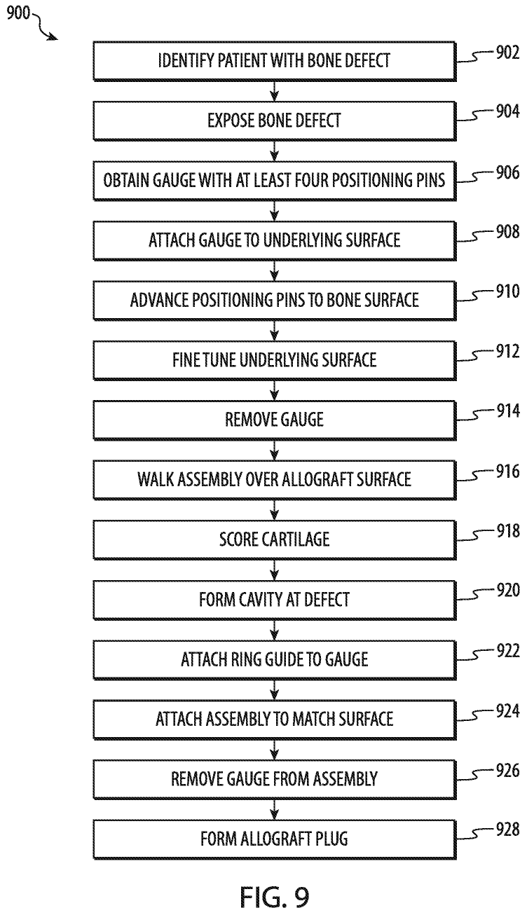

In more detail, FIG. 9 shows a flow chart of a method for transplanting osteoarticular allografts in accordance with embodiments herein 900. In operation 902, a patient in need of a surgical transplant to fill a bone defect is identified. Typical bone defects include those formed by trauma, infection, cancer and the like, and typically relates to condyle bone.

In operation 904, a surgeon (or other health care worker) makes an appropriate incision, and the bone surface defect exposed. In operation 906, a gauge is obtained having at least four positioning pins, each pin advanced at least 10 mm below the bottom surface of a central hub (at least 10 mm from the bottom surface of the central hub is necessary for positioning of a ring guide). As noted above, contour gauges can be of different sizes so as to relate to a particular defect, so that there is enough area to cover or prepare a surface profile for a particular patient's defect. In addition, contour gauges in accordance with embodiments herein may include 4, 5, 6, 7, 8, 9, 10 or more positioning pins, dependent on the level of accuracy required for determining a surface profile.

In operation 908, an engagement pin or steinmann pin is received in the alignment tube and advanced into the recipient condyle. The contour gauge is now attached to the bone surface having the bone defect. In typical embodiments, the steinmann pin is attached directly into the defect, and more typically, directly into the center of the defect. The 12 o'clock position is marked on the patient's condyle (note that any position of a clock face can be used, as long as it is consistently used throughout the procedure as the direction of orientation).

In operation 910, a properly prepared and attached contour gauge, placed over the bone defect, has its positioning pins advanced to settle on the condular surface of the bone. Positioning pins can be slidingly engaged with the central hub for advancement or can be advanced via a threaded engagement between the positioning pin and central hub. In typical embodiments, there are at least four, at least six or at least eight positioning pins advanced to the underlying bone surface. The positioning pins are always advanced to a distance from the central hub to allow for attachment of a ring guide, which is typically at least 10 mm.

In operation 912, the positioning pins can be further advanced out of the central hub to provide a “fine-tuned” negative surface profile of the underlying bone surface. Fine-tuning can include use of a longitudinal groove on embodiments of the central hub of the contour gauge. In this embodiment, the groove is oriented toward the 12 o'clock position to provide an overall orientation for the contour gauge. A positioning pin at the 12 o'clock position on the central hub is typically advanced until it touches the condyle surface. Once the 12 o'clock positioning pin is correctly engaged and oriented, the other positioning pins are advanced until all pins touch the condyle surface. In some cases the pins may be advanced and retracted until the proper underlying profile is attained.

In operation 914, the gauge having the surface profile set, is removed from the defect site, leaving the attachment pin in the defect. In operation 916, the profile set contour gauge is now walked over the surface of the donor tissue (allograft). A proper contour surface match is one where all positioning pins are touching the allograft surface. However, in some cases, ⅞, ⅚ or ¾ pins may touch. The surgeon uses the contour gauge to identify the best possible surface contour match available under the conditions, and in some cases, may walk the contour gauge over more than one allograft source. However, once a match is identified, the 12 o'clock and 6 o'clock positioning pin positions are marked on the allograft surface. As noted above, as long as consistent markers are used, other points around a clock face may be used, for example 3 o'clock and 9 o'clock.

In operation 918, a cutter/guide is then placed at the defect site to score the site using the attachment pin for orientation.

In operation 920, a flat blade, or other like instrument, is advanced to an appropriate depth to form a cavity at the defect site, such that the cavity is set to receive a properly prepared osteoarticular allograft. Depths at the defect site are noted at 12 o'clock, 3 o'clock, 6 o'clock and 9 o'clock. As noted above, other positions of the clock face can be used as long as the comparisons herein are consistent.

In operation 922, the contour gauge is loaded or engaged with a ring guide, such that the ring guide encompasses all of the gauge and positioning pins. The ring guide slides up against the lower or patient side surface of the contour gauge. The assembly is now positioned on the allograft at the previously marked locations. The positioning pins should rest against the allograft surface and the contour gauge assembly is in the correct location and orientation to match the patient's defect bone surface.

Optionally, in operation 924, the appropriate position on the allograft source having been determined, one or more short steinmann pins is drilled into the condyle through a perimeter hole of the ring guide to attach the ring guide to the allograft surface. The ring guide may also be secured by an articulating arm with a locking mechanism.

In operation 926, the contour gauge is removed from the ring guide, leaving the ring guide in place over the underlying bone surface.

In operation 928, the allograft is scored with the cutter/guide and the 12 o'clock position marked. A contour reamer and saw are positioned in the ring guide and advanced to the depths previously identified for the condyle defect. In some embodiments, the depth is set at about 18 to 22 mm depth, and more typically, about 20 mm of depth. The ring guide and pins are now removed, and the allograft cross-cut at the appropriate depth. In one embodiment, 12, 3, 6 and 9 depth measurements are taken from the patient condyle, and the allograft plug is trimmed to those depths. The plug is reduced and placed into the patient's bone cavity.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

What is claimed is:

1. A contour gauge for bone surface remodeling, comprising:
a central hub having:
a top surface; and
a bottom surface opposite the top surface; and
a plurality of positioning pins extending through a matching number of openings in the central hub, wherein:
the positioning pins transverse through the one or more openings in the central hub such that a distal end of the positioning pins can be independently extended from each other to contact a bone surface and provide a contour of the bone surface.

2. The contour gauge of claim 1, where an alignment tube is operatively attached to the top surface, the alignment tube creating an opening in the central hub extending form the top surface to the bottom surface of the central hub.

3. The contour gauge of claim 1, wherein the contour of the bone surface is defined by a distance of the central hub to the bone surface by each of the positioning pins.

4. The contour gauge of claim 2, further comprising an attachment pin, wherein the attachment pin is received in the alignment tube for operatively tethering the central hub to the bone surface.

5. The contour gauge of claim 1, wherein the plurality of positioning pins is from 4 to 8 in number.

6. A gauge, comprising:
a central hub capable of attaching to a bone surface; and
a series of contour measurement pins extending from the central hub, wherein:
when the central hub is attached to the bone surface, the contour measurement pins are capable of being fixedly oriented to map a contour of the bone surface.

7. The gauge of claim 6, wherein the series of contour measurement pins are positioned around a periphery of the central hub.

8. The gauge of claim 7, wherein the series of contour measurement pins further comprises one measurement pin positioned interior to the periphery of the central hub.

9. The gauge of claim 8, wherein the central hub has a circular or oval shape.

10. A method, comprising:
determining a first surface contour of a target bone defect;
excising the target bone defect having the first surface contour to form a target bone cavity;
matching the first surface contour to a second surface contour on a donor cadaver bone;
forming an allograft plug having the second surface contour from the donor cadaver bone; and
transplanting the allograft plug into the target bone cavity.

11. The method of claim 10, wherein the determining the first surface contour of the target bone defect is by use of a gauge that sets a negative surface profile for use in matching the first surface contour to the second surface contour.

12. The method of claim 11, wherein the negative surface profile set by the gauge has four or more surface contacts with the first surface contour.

13. The method of claim 12, wherein the negative surface profile set by the gauge has six surface contacts with the first surface contour.

14. A kit for transplanting a contour matched allograft plug to a condylar defect, comprising:
a gauge capable of matching a condylar defect surface contour to a donor bone surface contour;
a ring guide for marking the site for allograft plug removal;
a coring reamer for forming the allograft plug; and
trimming device for fine tuning the size of the allograft plug.

15. The kit of claim 14, further comprising two steinmann pins for attaching the ring guide to the allograft plug.

16. The kit of claim 14, wherein the gauge has a central hub capable of attaching to a bone surface; and a series of contour measurement pins extending from the central hub, wherein: when the central hub is attached to the bone surface, the contour measurement pins are capable of being fixedly oriented to map a contour of the bone surface.

17. A method for matching a first surface to a second surface, comprising:
sizing and making a negative surface contour of the first surface; and
screening the second surface with the negative surface contour until a match is found;
wherein, the match requires at least three points of substantial identity between the first surface and the second surface.

18. The method of claim 17, wherein the first surface has a first peripheral edge and the second surface has a second peripheral edge; and
wherein the match requires at least three points of substantial identity between the first peripheral edge and the second peripheral edge.

19. The method of claim 17, wherein the match requires at least four points of substantial identity between the first peripheral edge and the second peripheral edge.

20. The method of claim 17, wherein the match requires at least six points of substantial identity between the first peripheral edge and the second peripheral edge.

21. The method of claim 17, wherein the first surface is a surface of a bone defect and the second surface is a surface of an allograft.
